(19) United States
(12) Patent Application Publication
(10) Pub. No.: US 2026/0154810 A1
(43) Pub. Date: Jun. 4, 2026

(54) METHOD FOR ESTABLISHING ACUTE ISCHEMIC STROKE MODEL, METHOD FOR ASSESSING ACUTE ISCHEMIC STROKE AND ACUTE ISCHEMIC STROKE ASSESSING SYSTEM

(71) Applicant: China Medical University, Taichung City (TW)

(72) Inventors: Wei-Chun WANG, Taichung City (TW); Sheng-Ta TSAI, Taichung City (TW); Dang-Khoa NGUYEN, Taichung City (TW); Ching-Ting LIN, Taichung City (TW); Chien-Wei CHEN, Taichung City (TW); Yu-Wan YANG, Taichung City (TW); Ya-Lun WU, Taichung City (TW); Ming-Kuei LU, Taichung City (TW); Chon-Haw TSAI, Taichung City (TW); Kai-Cheng HSU, Hsinchu (TW); Shang-Yu CHIEN, Taichung City (TW)

(21) Appl. No.: 19/061,984

(22) Filed: Feb. 24, 2025

(30) Foreign Application Priority Data
Dec. 2, 2024 (TW) 113146654

(51) Int. Cl. G06T 7/00; A61B 6/03; A61B 6/50; G06V 10/774

(52) U.S. Cl. CPC G06T 7/0012; A61B 6/032; A61B 6/501; G06V 10/774; G06T 2207/10081; G06T 2207/10088; G06T 2207/20081; G06T 2207/30016; G06V 2201/031

(57) ABSTRACT

A method for assessing acute ischemic stroke includes following steps. An acute ischemic stroke model is provided. A plurality of target non-contrast computed tomography images and a plurality of target magnetic resonance images of a subject are obtained by the processor. A target area of each of the plurality of target non-contrast computed tomography images is labeled by the processor based on the one of the plurality of target magnetic resonance images corresponding thereto so as to obtain a plurality of labeled target non-contrast computed tomography images. The plurality of labeled target non-contrast computed tomography images are superimposed by the processor so as to obtain a 3D target non-contrast computed tomography image. The acute ischemic stroke model is performed by the processor to analyze a processed target non-contrast computed tomography image so as to obtain an acute ischemic stroke assessing result.

### RELATED APPLICATIONS

This application claims priority to Taiwan Application Serial Number 113146654, filed Dec. 2, 2024, which is herein incorporated by reference.

### BACKGROUND

### Technical Field

The present disclosure relates to a medical information analysis method and a medical information analysis system. More particularly, the present disclosure relates to a method for establishing an acute ischemic stroke model, a method for assessing acute ischemic stroke and an acute ischemic stroke assessing system.

### Description of Related Art

The occurrence of the stroke can seriously affect the physiological functions of the patient and even lead to death. The stroke can be categorized to the ischemic stroke and the hemorrhagic stroke, and the ischemic stroke is the most common. Currently, the brain computed tomography (CT) is the main diagnostic method for the stroke in clinical practice, and the brain computed tomography can be used to identify the type of stroke in patients.

In the diagnosis of the ischemic stroke, an additional time of about 15 minutes to 30 minutes is needed for applying the contrast agent to the subject and performing the brain scan to obtain the computed tomography perfusion (CTP) images so as to diagnose the ischemic stroke and evaluate the area thereof. However, the aforementioned method is time-consuming and prone to delay the time of the patient to receive the emergency treatment. Further, the non-contrast computed tomography (NCCT) is also applied clinically, and the non-contrast computed tomography can be used to diagnose the stroke directly without the use of the contrast agent. However, the non-contrast computed tomography can only be used to rule out cerebral hemorrhage, but cannot identify ischemic stroke.

Therefore, how to develop a method that can be used to rapidly and accurately assess the occurrence of the ischemic stroke has become a technical subject with clinical application value.

### SUMMARY

According to one aspect of the present disclosure, a method for establishing an acute ischemic stroke model includes following steps. An medical imaging database is obtained by a processor, wherein the medical imaging database includes a plurality of non-contrast computed tomography images and a plurality of magnetic resonance images of a plurality of reference subjects, and each of the plurality of non-contrast computed tomography images corresponds to one of the plurality of magnetic resonance images. A reference area of each of the plurality of non-contrast computed tomography images is labeled by the processor based on the one of the plurality of magnetic resonance images corresponding thereto so as to obtain a plurality of labeled non-contrast computed tomography images. The plurality of labeled non-contrast computed tomography images are superimposed by the processor so as to obtain a plurality of 3D non-contrast computed tomography images, and a reference 3D area of each of the plurality of 3D non-contrast computed tomography images is segmented by the processor so as to obtain a plurality of processed 3D non-contrast computed tomography images, wherein each of the plurality of 3D non-contrast computed tomography images corresponds to one of the reference subjects, and the reference 3D area includes the reference area of each of the plurality of non-contrast computed tomography images of the one of the reference subjects. A first deep-learning model is performed by the processor to train the plurality of processed 3D non-contrast computed tomography images to achieve a convergence so as to obtain the acute ischemic stroke model.

According to another aspect of the present disclosure, a method for assessing acute ischemic stroke includes following steps. The acute ischemic stroke model established by the method for establishing the acute ischemic stroke model according to the aforementioned aspect is provided. A plurality of target non-contrast computed tomography images and a plurality of target magnetic resonance images of a subject are obtained by the processor, wherein each of the plurality of target non-contrast computed tomography images corresponds to one of the plurality of target magnetic resonance images, and an acquisition time of the plurality of target non-contrast computed tomography images is the same as an acquisition time of the plurality of target magnetic resonance images. A target area of each of the plurality of target non-contrast computed tomography images is labeled by the processor based on the one of the plurality of target magnetic resonance images corresponding thereto so as to obtain a plurality of labeled target non-contrast computed tomography images. The plurality of labeled target non-contrast computed tomography images are superimposed by the processor so as to obtain a 3D target non-contrast computed tomography image, and a target 3D area of the 3D target non-contrast computed tomography image is segmented by the processor so as to obtain a processed target non-contrast computed tomography image, wherein the target 3D area includes the target area of each of the plurality of target non-contrast computed tomography images. The acute ischemic stroke model is performed by the processor to analyze the processed target non-contrast computed tomography image so as to obtain an acute ischemic stroke assessing result, wherein the acute ischemic stroke assessing result includes an ischemic area map within 24 hours after the acquisition time of the plurality of target non-contrast computed tomography images.

According to further another aspect of the present disclosure, an acute ischemic stroke assessing system includes a memory and a processor. The memory is for storing a plurality of target non-contrast computed tomography images and a plurality of target magnetic resonance images of a subject, wherein each of the plurality of target non-contrast computed tomography images corresponds to one of the plurality of target magnetic resonance images, and an acquisition time of the plurality of target non-contrast computed tomography images is the same as an acquisition time of the plurality of target magnetic resonance images. The processor is signally connected to the memory and includes a labeling model, a 3D image generation model, an image segmentation model and an acute ischemic stroke model. A target area of each of the plurality of target non-contrast computed tomography images is labeled by the labeling model based on one of the plurality of target magnetic resonance images corresponding thereto so as to obtain a plurality of labeled target non-contrast computed tomography images. The plurality of labeled target non-contrast computed tomography images are superimposed by the 3D image generation model so as to obtain a 3D target non-contrast computed tomography image. A target 3D area of the 3D target non-contrast computed tomography image is segmented by the image segmentation model so as to obtain a processed target non-contrast computed tomography image, and the target 3D area includes the target area of each of the plurality of target non-contrast computed tomography images. The acute ischemic stroke model is for analyzing the processed target non-contrast computed tomography image so as to obtain an acute ischemic stroke assessing result, wherein the acute ischemic stroke assessing result includes an ischemic area map within 24 hours after the acquisition time of the plurality of target non-contrast computed tomography images.

### BRIEF DESCRIPTION OF THE DRAWINGS

The present disclosure can be more fully understood by reading the following detailed description of the embodiment, with reference made to the accompanying drawings as follows:

FIG. 1 is a flow chart of a method for establishing an acute ischemic stroke model according to one embodiment of the present disclosure.

FIG. 2 is a flow chart of a method for assessing acute ischemic stroke according to another embodiment of the present disclosure.

FIG. 3 is a block diagram of an acute ischemic stroke assessing system according to further another embodiment of the present disclosure.

FIG. 4 shows the non-contrast computed tomography images of different subjects in the reference database of the present disclosure.

FIG. 5 shows the magnetic resonance images of different subjects in the reference database of the present disclosure.

FIG. 6 shows the ischemic area maps of different subjects obtained by the method for assessing acute ischemic stroke and the acute ischemic stroke assessing system of the present disclosure used to assess the acute ischemic stroke.

FIG. 7 shows the correlation diagram between the volume values of ischemic area obtained by the method for assessing acute ischemic stroke and the acute ischemic stroke assessing system of the present disclosure used to assess the acute ischemic stroke and the volume values of ischemic area in the ground truth status.

### DETAILED DESCRIPTION

The present disclosure will be further exemplified by the following specific embodiments. However, these practical details are used to describe how to implement the materials and methods of the present disclosure and are not necessary.

### [the Method for Establishing the Acute Ischemic Stroke Model of the Present Disclosure]

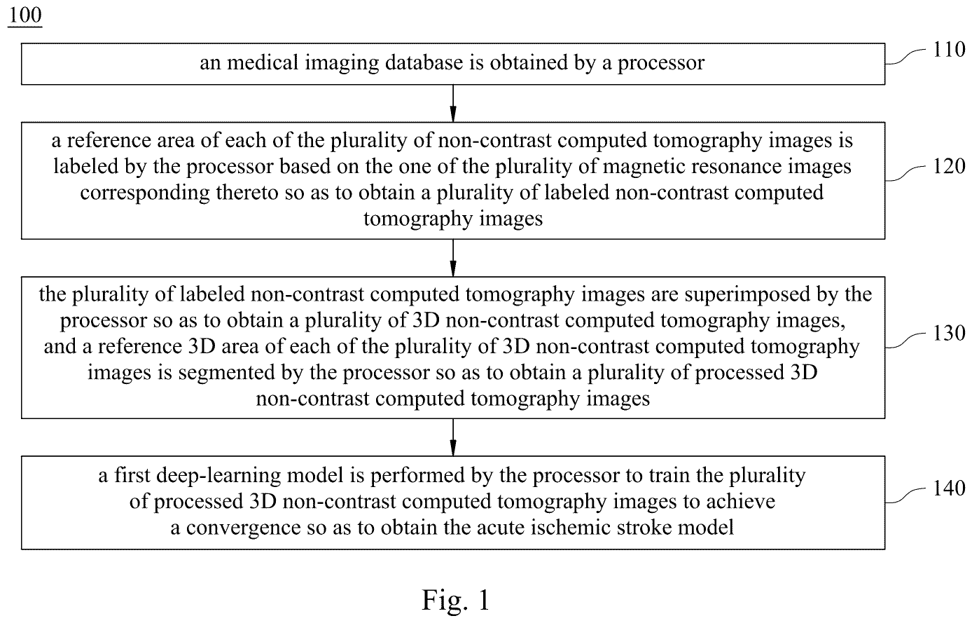

Reference is made to FIG. 1, which is a flow chart of a method 100 for establishing an acute ischemic stroke model according to one embodiment of the present disclosure. The method 100 for establishing the acute ischemic stroke model includes Step 110, Step 120, Step 130 and Step 140.

In Step 110, an medical imaging database is obtained by a processor, wherein the medical imaging database includes a plurality of non-contrast computed tomography (NCCT) images and a plurality of magnetic resonance images (MRIs) of a plurality of reference subjects, and each of the plurality of non-contrast computed tomography images corresponds to one of the plurality of magnetic resonance images. In detail, each of the reference subjects can include a plurality of non-contrast computed tomography images and a plurality of magnetic resonance images, wherein the non-contrast computed tomography images of each of the reference subjects can be a plurality of serial non-contrast computed tomography images within a finite depth region of a tissue, but the present disclosure is not limited thereto. Further, in the method 100 for establishing the acute ischemic stroke model, the non-contrast computed tomography image and the magnetic resonance image corresponding thereto should be obtained simultaneously from the electronic devices in the medical institution, so that the correspondence between the non-contrast computed tomography image and the magnetic resonance image corresponding thereto can be ensured. Furthermore, the tissue can be the brain tissue, and the finite depth region can be a 3D space of a target area within the brain tissue so as to identify the area in the brain tissue where the ischemia occurs.

In Step 120, a reference area of each of the plurality of non-contrast computed tomography images is labeled by the processor based on the one of the plurality of magnetic resonance images corresponding thereto so as to obtain a plurality of labeled non-contrast computed tomography images. In detail, each of the magnetic resonance images can include a lesion area, and the lesion area is obtained by analyzing the magnetic resonance image by a deep-learning model. Then, an area of the non-contrast computed tomography image corresponding to the lesion area of the magnetic resonance image is labeled so as to obtain the reference area of the non-contrast computed tomography image. Further, the deep-learning model used to analyze the magnetic resonance images can be a self-adapting framework for U-Net-Based medical image segmentation (nnU-Net), but the present disclosure is not limited thereto.

In Step 130, the plurality of labeled non-contrast computed tomography images are superimposed by the processor so as to obtain a plurality of 3D non-contrast computed tomography images, and a reference 3D area of each of the plurality of 3D non-contrast computed tomography images is segmented by the processor so as to obtain a plurality of processed 3D non-contrast computed tomography images, wherein each of the plurality of 3D non-contrast computed tomography image corresponds to one of the reference subjects, and the reference 3D area includes the reference area of each of the plurality of non-contrast computed tomography images of the one of the reference subjects. In detail, after the labeled non-contrast computed tomography images are superimposed, the reference areas, that are labeled based on the lesion areas of the magnetic resonance images and presented as 2D images, of the non-contrast computed tomography images will be superimposed, and the reference 3D area is formed by superimposing the plurality of reference areas of the plurality of non-contrast computed tomography images.

Further, the unit of the reference 3D area can be voxel, and the reference 3D area can be segmented out from the 3D non-contrast computed tomography image. Voxel is the smallest unit in the 3D space segmentation, and in response to the needs of different resolutions, the number of the segmentations in each dimension is also different. Thus, the accuracy of the analysis of the target area in medical can be enhanced, but the present disclosure is not limited thereto.

Further, in order to enhance the analysis efficiency of the acute ischemic stroke model established by the method 100 for establishing the acute ischemic stroke model of the present disclosure, the reference 3D area can be segmented and obtained from the 3D non-contrast computed tomography image by the segmenting methods such as random cropping and random flipping on three axes, but the present disclosure is not limited thereto.

Further, the size of each of the plurality of reference 3D areas can be adjusted by a standardized method so as to obtain the processed 3D non-contrast computed tomography images, and thus the interference signals of the method 100 for establishing the acute ischemic stroke model of the present disclosure can be reduced. In detail, the standardized method is used to adjust the size of each of the reference 3D areas after segmentation by the processor so as to make the sizes consistent. Thus, the plurality of processed 3D non-contrast computed tomography images can be obtained.

In Step 140, a first deep-learning model is performed by the processor to train the plurality of processed 3D non-contrast computed tomography images to achieve a convergence so as to obtain the acute ischemic stroke model. Further, the first deep-learning model can include a SwimUNETR deep-learning module, but the present disclosure is not limited thereto.

In detail, the SwimUNETR deep-learning module is a hierarchical vision transformer and is recently proposed to improve the efficiency of the self-attention computation. By the SwimUNETR deep-learning module, all of the processed 3D non-contrast computed tomography images can be highly correlated so as to enhance the accuracy of the result output by the acute ischemic stroke model. Further, the SwimUNETR deep-learning module can include an encoder and a decoder, and the encoder is connected to the decoder via skip connections at different resolutions of feature representations created by the encoder, wherein the decoder can be a decoder based on a convolution neural network, but the present disclosure is not limited thereto.

Further, the first deep-learning model can further include a normalization module, and the normalization module can be an uncertainty quantification module (UQM). In detail, the uncertainty quantification module can enhance the credibility and the efficiency of the acute ischemic stroke model trained and obtained based on the first deep-learning model, and the input source of the first deep-learning model can be analyzed through the probability distribution so as to output the assessing results with high reliability.

Therefore, by the methods that the reference area of each of the non-contrast computed tomography images is labeled, the reference areas of the non-contrast computed tomography images are superimposed and segmented, and the processed 3D non-contrast computed tomography images are trained by the first deep-learning model to obtain the acute ischemic stroke model, the acute ischemic stroke model with high reliability can be obtained by the method 100 for establishing the acute ischemic stroke model of the present disclosure. It is favorable for assessing the acute ischemic stroke, and the clinical application potential thereof is excellent.

### [the Method for Assessing Acute Ischemic Stroke of the Present Disclosure]

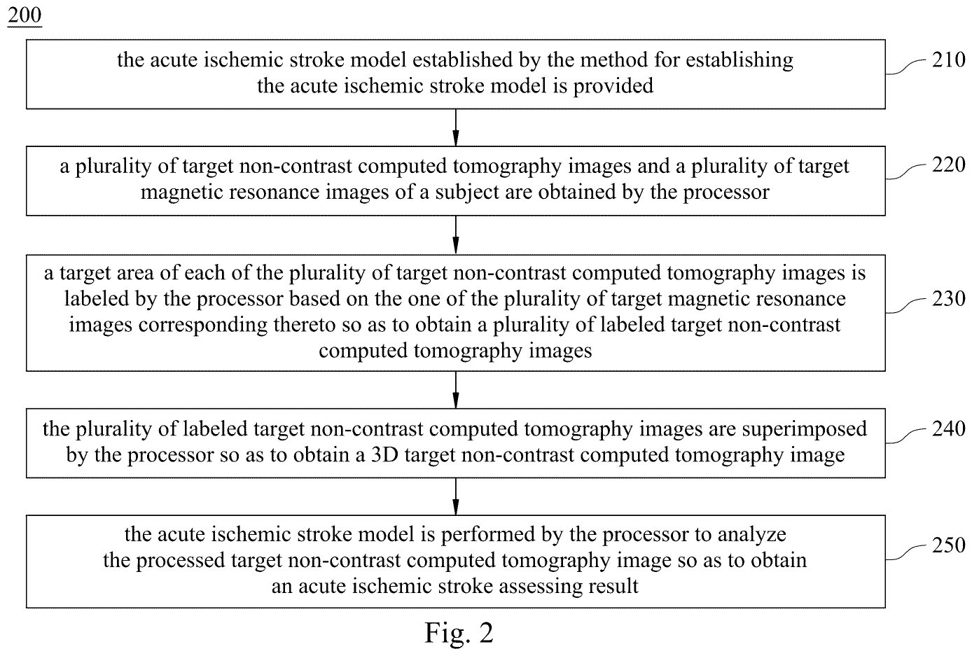

Reference is made to FIG. 2, which is a flow chart of a method 200 for assessing acute ischemic stroke according to another embodiment of the present disclosure. The method 200 for assessing acute ischemic stroke includes Step 210, Step 220, Step 230, Step 240 and Step 250.

In Step 210, the acute ischemic stroke model established by the method for establishing the acute ischemic stroke model is provided. In detail, the method for establishing the acute ischemic stroke model is the method 100 for establishing the acute ischemic stroke model of the present disclosure, and the acute ischemic stroke model is established by the method 100 for establishing the acute ischemic stroke model of the present disclosure. Thus, the details for establishing the acute ischemic stroke model are shown in the aforementioned paragraphs, and it will not be described again herein.

In Step 220, a plurality of target non-contrast computed tomography images and a plurality of target magnetic resonance images of a subject are obtained by the processor, wherein each of the plurality of target non-contrast computed tomography images corresponds to one of the plurality of target magnetic resonance images, and an acquisition time of the plurality of target non-contrast computed tomography images is the same as an acquisition time of the plurality of target magnetic resonance images. In detail, the plurality of target non-contrast computed tomography images and the plurality of target magnetic resonance images should be obtained simultaneously from the electronic devices in the medical institution, so that the correspondence between each of the target non-contrast computed tomography images and the one of the target magnetic resonance images corresponding thereto can be ensured, and the assessing accuracy of the method 200 for assessing acute ischemic stroke of the present disclosure can be enhanced.

Further, the target non-contrast computed tomography images can be a plurality of serial non-contrast computed tomography images within a finite depth region of a target tissue, and the target magnetic resonance images can be a plurality of serial magnetic resonance images within the finite depth region of the target tissue. Further, in the method 200 for assessing acute ischemic stroke, the target tissue can be the brain tissue, and the finite depth region can be a 3D space of a target area within the brain tissue so as to identify the area in the brain tissue where the ischemia occurs, but the present disclosure is not limited thereto.

In Step 230, a target area of each of the plurality of target non-contrast computed tomography images is labeled by the processor based on the one of the plurality of target magnetic resonance images corresponding thereto so as to obtain a plurality of labeled target non-contrast computed tomography images. In detail, each of the target magnetic resonance images can include a lesion area, and the lesion area is obtained by analyzing the target magnetic resonance image by a deep-learning model. Then, an area of the target non-contrast computed tomography image corresponding to the lesion area of the target magnetic resonance image is labeled so as to obtain the target area of the target non-contrast computed tomography image. Further, the deep-learning model used to analyze the target magnetic resonance images can be a self-adapting framework for U-Net-Based medical image segmentation, but the present disclosure is not limited thereto.

In Step 240, the plurality of labeled target non-contrast computed tomography images are superimposed by the processor so as to obtain a 3D target non-contrast computed tomography image, and a target 3D area of the 3D target non-contrast computed tomography image is segmented by the processor so as to obtain a processed target non-contrast computed tomography image, wherein the target 3D area includes the target area of each of the target non-contrast computed tomography images. In detail, after the labeled target non-contrast computed tomography images are superimposed, the target areas, that are labeled based on the lesion areas of the target magnetic resonance images and presented as 2D images, of the target non-contrast computed tomography images will be superimposed, and the target 3D area is formed by superimposing the plurality of target areas of the plurality of target non-contrast computed tomography images.

Further, the unit of the target 3D area can be voxel, the target 3D area can be segmented out from the 3D target non-contrast computed tomography image, and the target 3D area can be segmented and obtained from the 3D target non-contrast computed tomography image by the segmenting methods such as random cropping and random flipping on three axes, but the present disclosure is not limited thereto.

Further, the size of the target 3D area can be adjusted by the processor so as to make the size thereof consistent with those of the aforementioned reference 3D areas, so that the interference signals of the method 200 for assessing acute ischemic stroke of the present disclosure can be reduced.

In Step 250, the acute ischemic stroke model is performed by the processor to analyze the processed target non-contrast computed tomography image so as to obtain an acute ischemic stroke assessing result, wherein the acute ischemic stroke assessing result includes an ischemic area map within 24 hours after the acquisition time of the plurality of target non-contrast computed tomography images. Further, the acute ischemic stroke assessing result can further include a volume value of ischemic area and an ischemic area heat map. In detail, the golden hours of the acute ischemic stroke is within 24 hours from the time of discovery, and the earlier the disease is discovered, the better the treatment effect. Therefore, the method 200 for assessing acute ischemic stroke of the present disclosure can effectively output the ischemic area map within 24 hours after the acquisition time of the target non-contrast computed tomography images so as to enhance the clinical application potential of the method 200 for assessing acute ischemic stroke of the present disclosure.

Therefore, by analyzing the processed target non-contrast computed tomography image by the acute ischemic stroke model so as to obtain the acute ischemic stroke assessing result, it is favorable for the method 200 for assessing acute ischemic stroke of the present disclosure to facilitate diagnosing early whether the subject suffers from the acute ischemic stroke or not and assessing the severity of the acute ischemic stroke, and thus the clinical difficulty in diagnosing the acute ischemic stroke in the early stage can be solved.

### [the Acute Ischemic Stroke Assessing System of the Present Disclosure]

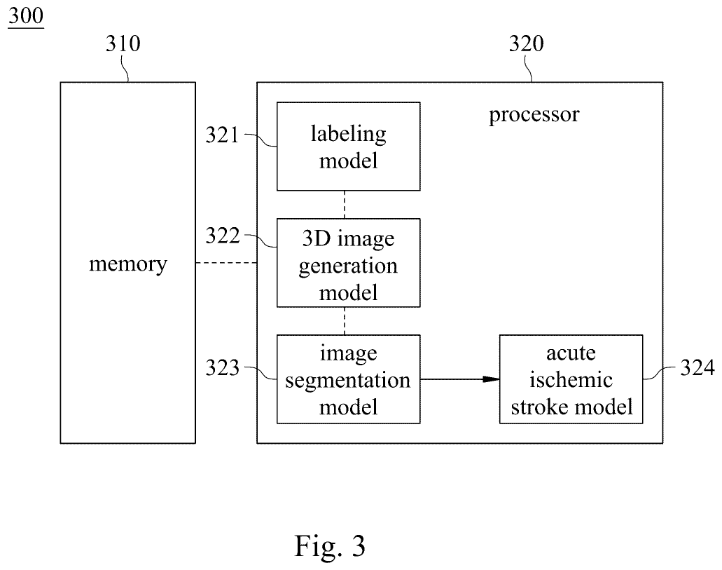

Reference is made to FIG. 3, which is a block diagram of an acute ischemic stroke assessing system 300 according to further another embodiment of the present disclosure. The acute ischemic stroke assessing system 300 includes a memory 310 and a processor 320, wherein the acute ischemic stroke assessing system 300 is for performing the method 200 for assessing acute ischemic stroke of the present disclosure, and the detail steps thereof are the same as those of the method 200 for assessing acute ischemic stroke of the present disclosure, and it will not be described again herein.

The memory 310 is for storing a plurality of target non-contrast computed tomography images and a plurality of target magnetic resonance images of a subject, wherein each of the plurality of target non-contrast computed tomography images corresponds to one of the plurality of target magnetic resonance images, and an acquisition time of the plurality of target non-contrast computed tomography images is the same as an acquisition time of the plurality of target magnetic resonance images. In detail, the plurality of target non-contrast computed tomography images and the plurality of target magnetic resonance images should be obtained simultaneously from the electronic devices in the medical institution, so that the correspondence between each of the target non-contrast computed tomography images and the one of the target magnetic resonance images corresponding thereto can be ensured, and the assessing accuracy of the acute ischemic stroke assessing system 300 of the present disclosure can be enhanced.

In detail, the target non-contrast computed tomography images can be a plurality of serial non-contrast computed tomography images within a finite depth region of a target tissue, and the target magnetic resonance images can be a plurality of serial magnetic resonance images within the finite depth region of the target tissue. Further, in the acute ischemic stroke assessing system 300, the target tissue can be the brain tissue, and the finite depth region can be a 3D space of a target area within the brain tissue so as to identify the area in the brain tissue where the ischemia occurs, but the present disclosure is not limited thereto.

The processor 320 is signally connected to the memory 310, and the processor 320 includes a labeling model 321, a 3D image generation model 322, an image segmentation model 323 and an acute ischemic stroke model 324.

A target area of each of the plurality of target non-contrast computed tomography images is labeled by the labeling model 321 based on one of the plurality of target magnetic resonance images corresponding thereto so as to obtain a plurality of labeled target non-contrast computed tomography images. In detail, each of the target magnetic resonance images can include a lesion area, and the lesion area is obtained by analyzing the target magnetic resonance image by a deep-learning model. Then, an area of the target non-contrast computed tomography image corresponding to the lesion area of the target magnetic resonance image is labeled so as to obtain the target area of the target non-contrast computed tomography image. Further, the deep-learning model used to analyze the target magnetic resonance images can be a self-adapting framework for U-Net-Based medical image segmentation, but the present disclosure is not limited thereto.

The plurality of labeled target non-contrast computed tomography images are superimposed by the 3D image generation model 322 so as to obtain a 3D target non-contrast computed tomography image. In detail, after the labeled target non-contrast computed tomography images are superimposed, the target areas, that are labeled based on the lesion areas of the target magnetic resonance images and presented as 2D images, of the target non-contrast computed tomography images will be superimposed, and the target 3D area is formed by superimposing the plurality of target areas of the plurality of target non-contrast computed tomography images.

A target 3D area of the 3D target non-contrast computed tomography image is segmented by the image segmentation model 323 so as to obtain a processed target non-contrast computed tomography image, and the target 3D area includes the target area of each of the plurality of target non-contrast computed tomography images.

Further, the unit of the target 3D area can be voxel, the target 3D area can be segmented out from the 3D target non-contrast computed tomography image, and the target 3D area can be segmented and obtained from the 3D target non-contrast computed tomography image by the segmenting methods such as random cropping and random flipping on three axes, but the present disclosure is not limited thereto.

Further, the size of the target 3D area can be adjusted by the image segmentation model 323 so as to make the size thereof consistent with those of the aforementioned reference 3D areas, so that the interference signals of the acute ischemic stroke assessing system 300 of the present disclosure can be reduced.

The acute ischemic stroke model 324 is for analyzing the processed target non-contrast computed tomography image so as to obtain an acute ischemic stroke assessing result, wherein the acute ischemic stroke assessing result includes an ischemic area map within 24 hours after the acquisition time of the plurality of target non-contrast computed tomography images. Further, the acute ischemic stroke assessing result can further include a volume value of ischemic area and an ischemic area heat map. Therefore, the acute ischemic stroke assessing system 300 of the present disclosure can effectively output the ischemic area map within 24 hours after the acquisition time of the target non-contrast computed tomography images so as to enhance the clinical application potential of the acute ischemic stroke assessing system 300 of the present disclosure.

Further, the acute ischemic stroke model 324 is obtained by training a plurality of non-contrast computed tomography images and a plurality of magnetic resonance images of a plurality of reference subjects by a first deep-learning model, and the first deep-learning model can include a SwimUNETR deep-learning module and a normalization module, wherein the normalization module can be an uncertainty quantification module. Furthermore, the establishing details of the acute ischemic stroke model 324 and the architecture of the first deep-learning model are the same as those of the method 100 for establishing the acute ischemic stroke model of FIG. 1 and are described in the aforementioned paragraphs, so that the same details will not be described again herein.

Therefore, by the arrangements of the acute ischemic stroke assessing system 300 of the present disclosure that the processor 320 is signally connected to the memory 310, and the processor 320 includes the labeling model 321, the 3D image generation model 322, the image segmentation model 323 and the acute ischemic stroke model 324, the plurality of target non-contrast computed tomography images and the plurality of target magnetic resonance images can be preprocessed by the labeling model 321, the 3D image generation model 322 and the image segmentation model 323 and then analyzed by the acute ischemic stroke model 324 so as to obtain the acute ischemic stroke assessing result within 24 hours after the occurrence of the acute ischemic stroke of the subject. Hence, it is favorable for diagnosing early the acute ischemic stroke, and the clinical difficulty in diagnosing the acute ischemic stroke in the early stage can be solved.

### EXAMPLE

### 1. Reference Database

In the example, the non-contrast computed tomography images and the magnetic resonance images are obtained from 317 subjects with different degrees of acute ischemic stroke at China Medical University Hospital, wherein the non-contrast computed tomography images and the magnetic resonance images of 237 subjects are served as the training dataset of the method for establishing the acute ischemic stroke model of the present disclosure, and the non-contrast computed tomography images and the magnetic resonance images of the remaining 80 subjects are served as the testing dataset of the method for assessing acute ischemic stroke of the present disclosure. The acquisition time of the non-contrast computed tomography images and the acquisition time of the magnetic resonance images of each of the subject are the same, and the diffusion-weighted imaging (DWI) of the magnetic resonance images is used as the ground truth status in the following tests.

The following tests are performed by the acute ischemic stroke model established by the method for establishing the acute ischemic stroke model of the present disclosure along with the method for assessing acute ischemic stroke and the acute ischemic stroke assessing system of the present disclosure so as to assess the accuracy of the method for assessing acute ischemic stroke and the acute ischemic stroke assessing system of the present disclosure used to assess the acute ischemic stroke. The acute ischemic stroke model of the present disclosure can be established by the method 100 for establishing the acute ischemic stroke model, and the method for assessing acute ischemic stroke of the present disclosure can be the method 200 for assessing acute ischemic stroke. Further, the acute ischemic stroke assessing system of the present disclosure can be the acute ischemic stroke assessing system 300. Thus, the same details are shown in the aforementioned paragraphs, and it will not be described again herein.

### 2. Establishing the Acute Ischemic Stroke Model of the Present Disclosure

In Example 1 of the present test, the model training is performed by the SwimUNETR deep-learning module as the main model and combined with the uncertainty quantification module so as to establish the acute ischemic stroke model of the present disclosure.

Further, the present test further includes Comparative example 1, Comparative example 2, Comparative example 3 and Comparative example 4, wherein Comparative example 1 uses the 3D UNet to train the training dataset, Comparative example 2 uses the 3D UNETR to train the training dataset, Comparative example 3 uses the 3D UNet++ to train the training dataset, and Comparative example 4 uses the SwimUNETR deep-learning module to train the training dataset, but Comparative example 4 is without the uncertainty quantification module. Accordingly, the models of Comparative example 1 to Comparative example 4 are established, and the 5-fold cross-validation is used to assess the performance among the models of Example 1, Comparative example 1, Comparative example 2, Comparative example 3 and Comparative example 4.

Reference is made to Table 1, which shows the results of the acute ischemic stroke model of the present disclosure used to train the training dataset. In Table 1, “Dice” represents the Dice score, “SEN” represents the pixel-wise sensitivity, and “SPE” represents the pixel-wise specificity.

TABLE 1

| | Dice | SEN | SPE |
|---|---|---|---|
| Comparative example 1 | 0.456 | 0.578 | 0.993 |
| Comparative example 2 | 0.399 | 0.576 | 0.991 |
| Comparative example 3 | 0.449 | 0.595 | 0.992 |
| Comparative example 4 | 0.452 | 0.601 | 0.992 |
| Example 1 | 0.467 | 0.659 | 0.991 |

As shown in Table 1, based on the calculation of the 5-fold cross-validation, the Dice score of the acute ischemic stroke model of Example 1 is 46.7%, the pixel-wise sensitivity thereof is 65.9%, and the pixel-wise specificity thereof is 99.1%. Compared with Comparative example 1 to Comparative example 4, the acute ischemic stroke model of Example 1 has better performance and efficiency. Further, with the assistance of the uncertainty quantification module, the pixel-wise sensitivity of the acute ischemic stroke model of Example 1 is 6% higher than the model of Comparative example 4 which lacks the uncertainty quantification module, and it shows that the uncertainty quantification module can enhance the training efficiency of the acute ischemic stroke model of the present disclosure.

### 3. Analysis of the Results of the Acute Ischemic Stroke Model of the Present Disclosure Used to Assess the Ischemic Area

In the present test, the acute ischemic stroke model of Example 1 is used to analyze the plurality of non-contrast computed tomography images and the plurality of magnetic resonance images of the training dataset, and the acute ischemic stroke assessing result obtained there from is further analyzed, wherein the acute ischemic stroke assessing result includes the ischemic area map and the volume value of ischemic area within 24 hours after the acquisition time of the non-contrast computed tomography images of the training dataset.

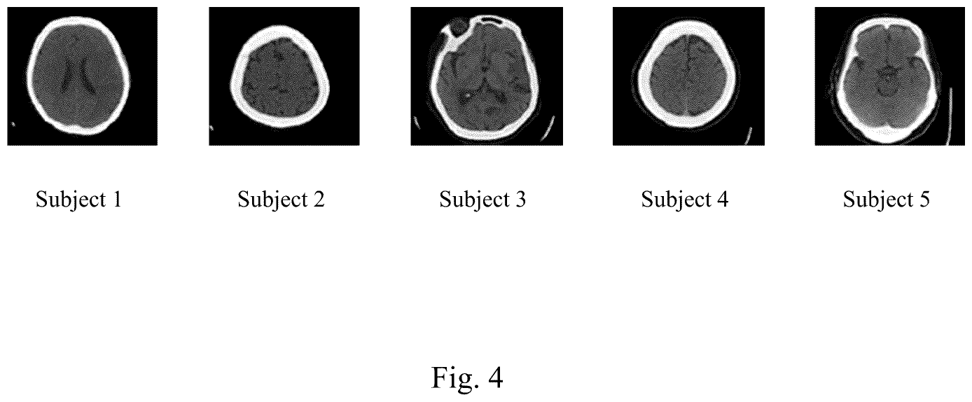

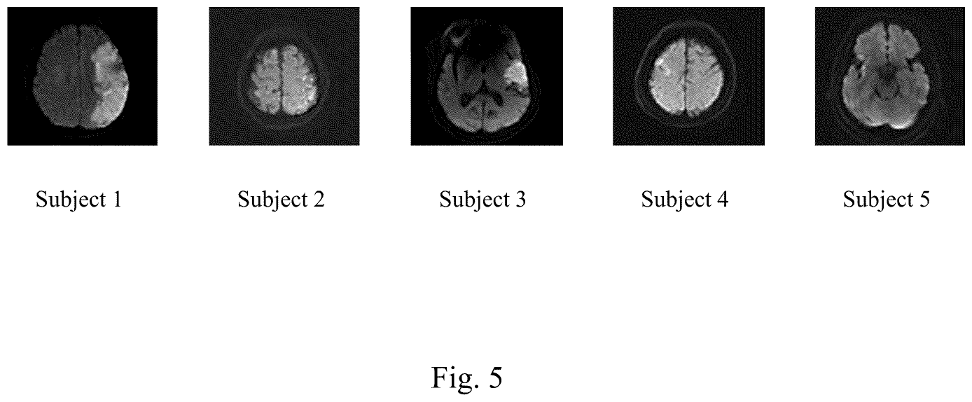

Reference is made to FIG. 4 and FIG. 5, wherein FIG. 4 shows the non-contrast computed tomography images of different subjects in the reference database of the present disclosure, and FIG. 5 shows the magnetic resonance images of different subjects in the reference database of the present disclosure. As shown in FIG. 5, the brighter area in each of the magnetic resonance images of Subject 1 to Subject 5 represents the area where the ischemic truly occurs, and the non-contrast computed tomography images of Subject 1 to Subject 5 of FIG. 4 are respectively labeled based on the ischemic areas shown in FIG. 5 by the labeling model of the processor and then analyzed by the acute ischemic stroke model of Example 1.

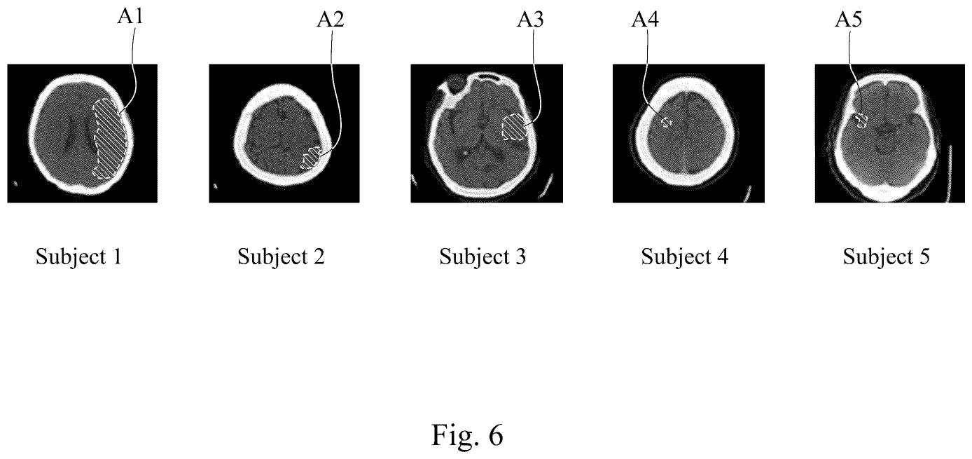

Reference is made to FIG. 6, which shows the ischemic area maps of different subjects obtained by the method for assessing acute ischemic stroke and the acute ischemic stroke assessing system of the present disclosure used to assess the acute ischemic stroke. As shown in FIG. 6, the area outlined by the white slashes in each of the ischemic area maps represents the ischemic area, wherein the ischemic area A1 of Subject 1, the ischemic area A2 of Subject 2, the ischemic area A3 of Subject 3, the ischemic area A4 of Subject 4 and the ischemic area A5 of Subject 5 approximately the same as the true ischemic areas in the target magnetic resonance images of FIG. 5, respectively. It shows that the accuracy of the positions of the ischemic areas obtained by the analysis of the acute ischemic stroke model of the present disclosure is excellent, and it is favorable for solving the difficulty in diagnosing the acute ischemic stroke in the early stage.

### 4. Analysis of the Results of the Acute Ischemic Stroke Model of the Present Disclosure Used to Assess the Volume Value of Ischemic Area

In the present test, the data of the testing dataset is classified based on different volume values of ischemic area into six testing groups, namely (1) the volume value of ischemic area is greater than 0 mL; (2) the volume value of ischemic area is greater than 5 mL; (3) the volume value of ischemic area is greater than 10 mL; (4) the volume value of ischemic area is greater than 30 mL; (5) the volume value of ischemic area is greater than 50 mL; and (6) the volume value of ischemic area is greater than 70 mL. The six testing groups are respectively analyzed by the acute ischemic stroke model of Example 1. As shown in the analysis results of the six testing groups, as the volume value of ischemic area increases, the Dice score thereof is higher, and the Dice score of the testing group with the volume value of ischemic area greater than 70 mL reaches 65.54%.

Further, all of the images of the testing dataset are divided into two testing groups with the volume value of ischemic area of 70 mL as the dividing point in the present test, and the images of the two testing groups are respectively analyzed by the acute ischemic stroke model of Example 1. As shown in the analysis results thereof, the accuracy is 81.25%, the area under the receiver operating characteristic curve (AUC) is 87.07%, the sensitivity is 100%, and the specificity is 74.14%.

As shown above, the acute ischemic stroke model of the present disclosure can be applied to identify the volume values of ischemic area and preliminary assess the status of the acute ischemic stroke of the subjects based on the target non-contrast computed tomography images thereof, so that the degrees of acute ischemic stroke of the subjects can be assessed, and the clinical application potential thereof is excellent.

Furthermore, all of the images of the testing dataset are classified based on the volume values of ischemic area and the time from the occurrence of the acute ischemic stroke to the acquisition time of the testing dataset (onset-to-CT time, “OCT time” hereafter) so as to obtain different testing groups. The images of the testing dataset are classified based on the volume values of ischemic area to obtain three testing groups, namely (1) the volume value of ischemic area is between 0 mL to 40 mL; (2) the volume value of ischemic area is between 40 mL to 100 m; and (3) the volume value of ischemic area is greater than 100 mL. The images of the testing dataset are classified based on the OCT time to obtain four testing groups, namely (1) the acquisition time of the testing dataset is 0 hour to 3 hours after the acute ischemic stroke occurs; (2) the acquisition time of the testing dataset is 3 hours to 6 hours after the acute ischemic stroke occurs; (3) the acquisition time of the testing dataset is 6 hours to 24 hours after the acute ischemic stroke occurs; and (4) the acquisition time of the testing dataset is 24 hours after the acute ischemic stroke occurs. The aforementioned testing groups are respectively analyzed by the acute ischemic stroke model of Example 1.

Reference is made to Table 2, which shows the Dice scores of the testing groups classified based on the volume values of ischemic area and the OCT times.

TABLE 2

| | | | OTC time | | | |
|---|---|---|---|---|---|---|
| | | | 0-3 hours | 3-6 hours | 6-24 hours | >24 hours |
| Volume | 0-40 | mL | 0.35 | 0.36 | 0.31 | 0.37 |
| value of | | | | | | |
| ischemic | 40-100 | mL | 0.54 | 0.59 | 0.58 | N/A |
| area | 100 | mL | 0.64 | 0.67 | 0.80 | N/A |

As shown in Table 2, when the volume value of ischemic area is greater than 40 mL and the OTC time is between 0 hours to 24 hours, the Dice scores thereof are greater than 0.5, and it shows that the acute ischemic stroke model of the present disclosure can be applied to diagnose the acute ischemic stroke in the early stage.

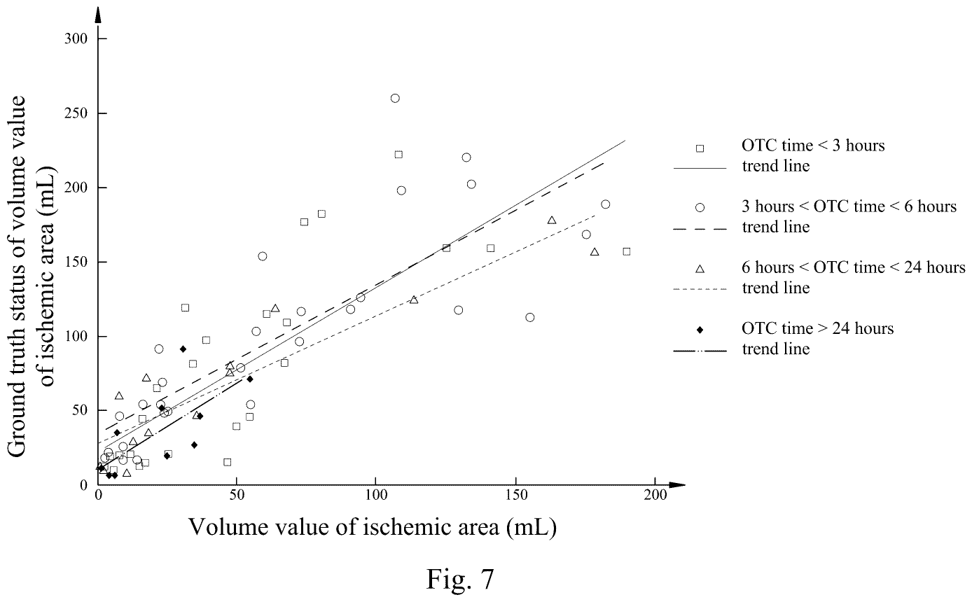

Reference is made to FIG. 7, which shows the correlation diagram between the volume values of ischemic area obtained by the method for assessing acute ischemic stroke and the acute ischemic stroke assessing system of the present disclosure used to assess the acute ischemic stroke and the volume values of ischemic area in the ground truth status. In order to illustrate the relation between the volume values of ischemic area obtained by the acute ischemic stroke model of the present disclosure and the ground truth status of the volume values of ischemic area obtained from the magnetic resonance images, in FIG. 7, the volume values of ischemic area obtained by the acute ischemic stroke model of the present disclosure and the ground truth status of the volume values of ischemic area obtained from the magnetic resonance images are compared with each other along with the OTC times shown in Table 2, wherein the ground truth status of the volume values of ischemic area are the volume value of ischemic area of the magnetic resonance images.

As shown in the trend lines of FIG. 7, there is a positive relationship between the volume values of ischemic area and the ground truth status of the volume values of ischemic area when the OTC time is less than 24 hours. On the contrary, when the OTC time is greater than 24 hours, the relation between the volume values of ischemic area and the ground truth status of the volume values of ischemic area are low. Accordingly, it shows that the acute ischemic stroke model of the present disclosure can effectively assess the volume of ischemic area when the OTC time is less than 24 hours, and the volume values of ischemic area thereof are corresponding to the ground truth status of the ischemic area, so that the clinical application potential thereof is excellent.

Therefore, the method for establishing an acute ischemic stroke model, the method for assessing acute ischemic stroke and the acute ischemic stroke assessing system of the present disclosure can accurately and rapidly assess the ischemic area of the subject and estimate the volume value of ischemic area of the subject within 24 hours after the acute ischemic stroke occurs, so that it is favorable for early diagnosing the acute ischemic stroke and shortening the treatment time of the patients, and the clinical application potential thereof is excellent.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A method for establishing an acute ischemic stroke model, comprising:
obtaining an medical imaging database by a processor, wherein the medical imaging database comprises a plurality of non-contrast computed tomography images and a plurality of magnetic resonance images of a plurality of reference subjects, and each of the plurality of non-contrast computed tomography images corresponds to one of the plurality of magnetic resonance images;
labeling a reference area of each of the plurality of non-contrast computed tomography images by the processor based on the one of the plurality of magnetic resonance images corresponding thereto so as to obtain a plurality of labeled non-contrast computed tomography images;
superimposing the plurality of labeled non-contrast computed tomography images by the processor so as to obtain a plurality of 3D non-contrast computed tomography images, and segmenting a reference 3D area of each of the plurality of 3D non-contrast computed tomography images by the processor so as to obtain a plurality of processed 3D non-contrast computed tomography images, wherein each of the plurality of 3D non-contrast computed tomography images corresponds to one of the reference subjects, and the reference 3D area comprises the reference area of each of the plurality of non-contrast computed tomography images of the one of the reference subjects; and
performing a first deep-learning model by the processor to train the plurality of processed 3D non-contrast computed tomography images to achieve a convergence so as to obtain the acute ischemic stroke model.

2. The method for establishing the acute ischemic stroke model of claim 1, wherein the first deep-learning model comprises a SwimUNETR deep-learning module.

3. The method for establishing the acute ischemic stroke model of claim 2, wherein the first deep-learning model further comprises a normalization module.

4. The method for establishing the acute ischemic stroke model of claim 3, wherein the normalization module is an uncertainty quantification module.

5. A method for assessing acute ischemic stroke, comprising:
providing the acute ischemic stroke model established by the method for establishing the acute ischemic stroke model of claim 1;
obtaining a plurality of target non-contrast computed tomography images and a plurality of target magnetic resonance images of a subject by the processor, wherein each of the plurality of target non-contrast computed tomography images corresponds to one of the plurality of target magnetic resonance images, and an acquisition time of the plurality of target non-contrast computed tomography images is the same as an acquisition time of the plurality of target magnetic resonance images;
labeling a target area of each of the plurality of target non-contrast computed tomography images by the processor based on the one of the plurality of target magnetic resonance images corresponding thereto so as to obtain a plurality of labeled target non-contrast computed tomography images;
superimposing the plurality of labeled target non-contrast computed tomography images by the processor so as to obtain a 3D target non-contrast computed tomography image, and segmenting a target 3D area of the 3D target non-contrast computed tomography image by the processor so as to obtain a processed target non-contrast computed tomography image, wherein the target 3D area comprises the target area of each of the plurality of target non-contrast computed tomography images; and
performing the acute ischemic stroke model by the processor to analyze the processed target non-contrast computed tomography image so as to obtain an acute ischemic stroke assessing result, wherein the acute ischemic stroke assessing result comprises an ischemic area map within 24 hours after the acquisition time of the plurality of target non-contrast computed tomography images.

6. The method for assessing acute ischemic stroke of claim 5, wherein the plurality of target non-contrast computed tomography images are a plurality of serial non-contrast computed tomography images within a finite depth region of a target tissue, and the plurality of target magnetic resonance images are a plurality of serial magnetic resonance images within the finite depth region of the target tissue.

7. The method for assessing acute ischemic stroke of claim 5, wherein the acute ischemic stroke assessing result further comprises a volume value of ischemic area and an ischemic area heat map.

8. An acute ischemic stroke assessing system, comprising:
a memory for storing a plurality of target non-contrast computed tomography images and a plurality of target magnetic resonance images of a subject, wherein each of the plurality of target non-contrast computed tomography images corresponds to one of the plurality of target magnetic resonance images, and an acquisition time of the plurality of target non-contrast computed tomography images is the same as an acquisition time of the plurality of target magnetic resonance images; and
a processor signally connected to the memory and comprising:
a labeling model, wherein a target area of each of the plurality of target non-contrast computed tomography images is labeled by the labeling model based on one of the plurality of target magnetic resonance images corresponding thereto so as to obtain a plurality of labeled target non-contrast computed tomography images;
a 3D image generation model, wherein the plurality of labeled target non-contrast computed tomography images are superimposed by the 3D image generation model so as to obtain a 3D target non-contrast computed tomography image;
an image segmentation model, wherein a target 3D area of the 3D target non-contrast computed tomography image is segmented by the image segmentation model so as to obtain a processed target non-contrast computed tomography image, and the target 3D area comprises the target area of each of the plurality of target non-contrast computed tomography images; and
an acute ischemic stroke model for analyzing the processed target non-contrast computed tomography image so as to obtain an acute ischemic stroke assessing result, wherein the acute ischemic stroke assessing result comprises an ischemic area map within 24 hours after the acquisition time of the plurality of target non-contrast computed tomography images.

9. The acute ischemic stroke assessing system of claim 8, wherein the acute ischemic stroke model is established by training a plurality of non-contrast computed tomography image and a plurality of magnetic resonance images of a plurality of reference subjects by a first deep-learning model.

10. The acute ischemic stroke assessing system of claim 9, wherein the first deep-learning model comprises a SwimUNETR deep-learning module.

11. The acute ischemic stroke assessing system of claim 10, wherein the first deep-learning model further comprises a normalization module.

12. The acute ischemic stroke assessing system of claim 11, wherein the normalization module is an uncertainty quantification module.

13. The acute ischemic stroke assessing system of claim 8, wherein the plurality of target non-contrast computed tomography images are a plurality of serial non-contrast computed tomography images within a finite depth region of a target tissue, and the plurality of target magnetic resonance images are a plurality of serial magnetic resonance images within the finite depth region of the target tissue.

14. The acute ischemic stroke assessing system of claim 8, wherein the acute ischemic stroke assessing result further comprises a volume value of ischemic area and an ischemic area heat map.
